(19) United States
(12) Patent Application Publication
(10) Pub. No.: US 2011/0190165 A1
(43) Pub. Date: Aug. 4, 2011

(54) HIGH THROUGHPUT METHODS OF IDENTIFYING NEUTRAL LIPID SYNTHASES

(76) Inventors: Randall Weselake, Edmonton (CA); Rodrigo Siloto, Edmonton (CA); Martin Reusksa, Edmonton (CA)

(21) Appl. No.: 12/993,055

(22) PCT Filed: May 19, 2009
(86) PCT No.: PCT/CA09/00678

§ 371 Date: Apr. 21, 2011

Related U.S. Application Data
(60) Provisional application No. 61/053,849, filed on May 16, 2008.

(51) Int. Cl. C40B 30/08; C12Q 1/48

(52) U.S. Cl. 506/11; 435/15

(57) ABSTRACT

The present invention relates to high throughput methods of identifying neutral lipid synthases. The invention includes a method of positively selecting yeast cells expressing recombinant neutral lipid synthases, and quantifying the enzyme activities of the recombinant neutral lipid synthases using a fluorescence in situ assay.

### FIELD OF THE INVENTION

The present invention relates to high throughput methods of identifying neutral lipid synthases.

### BACKGROUND OF THE INVENTION

Triacylglycerol (TAG) is an acyl ester of glycerol which represents the most efficient form of stored energy in most eukaryotes and some prokaryotes. The energy of oxidation of the acyl chains is much higher than the energy stored by the same mass of carbohydrates or proteins. Since TAG is stored into lipid droplets without the need for water, osmolarity is not increased. Alternatively, the acyl chains can be esterified to sterols, particularly steryl esters (SE), which serve a similar function. Accumulation of unesterified fatty acids in the cell may destabilize membranes; however, conjugation of unesterified fatty acids with glycerol and sterols may prevent such cytotoxic effects. Both TAG and SE are considered to be neutral lipids.

TAG biosynthesis occurs mainly in the endoplasmic reticulum of the cell using acyl-CoA and sn-glycerol-3-phosphate as primary substrates. Biosynthesis of TAG is effected through a biochemical process generally known as the Kennedy pathway which involves the sequential transfer of fatty acids from acyl-CoAs to the glycerol backbone (acyl-CoA-dependent acylation). The pathway starts with the acylation of sn-glycerol-3-phosphate to form lysophosphatidic acid through the action of sn-glycerol-3-phosphate acyltransferase. The second acylation is catalyzed by lysophosphatidic acid acyltransferase, leading to the formation of phosphatidic acid which is dephosphorylated by phosphatidate phosphatase 1 to form sn-1,2-diacylglycerol. The final acylation is catalyzed by diacylglycerol acyltransferase (DGAT; EC 2.3.1.20). The DGAT enzyme catalyzes the transfer of the acyl group from acyl-coenzymeA (acyl-CoA) donor to a sn-1,2-diacylglycerol, producing CoA and TAG. In contrast, TAG synthesis catalyzed by phospholipid:diacylglycerol acyltransferase (PDAT, EC 2.3.1.158) is acyl-CoA-independent and uses phospholipids as acyl donors and DAG as acceptor (Lung et al., 2006). Other uncharacterized TAG synthase enzymes can exist in nature. The TAG synthases DGAT and PDAT are membrane-bound enzymes located in endoplasmic reticulum (ER), which complicates their purification to homogeneity and hampers structural studies which may provide a greater understanding of these enzymes.

The final step of SE formation is accomplished in two different ways (Czabany et al., 2007). The first reaction, which is catalyzed by Acyl-coenzyme A:cholesterol acyltransferase (ACAT, EC2.3.1.26), uses sterol and acyl-CoA as substrates. The second reaction is acyl-CoA-independent and is catalyzed by lecithin:cholesterol acyltransferase (LCAT, EC 2.3.1.43) which utilizes phospholipids as alkyl donors.

In mammals, biosynthesis of TAG and SE functions in a number of homeostatic processes, including absorption of dietary fatty acids, energy storage in muscle and adipose tissues, and milk fat production (Farese et al., 2000). Excessive accumulation of TAG and SE contributes to obesity, hypertriglyceridemia and atherosclerosis. In attempt to prevent or treat these adverse conditions, therapeutic approaches have been directed to appetite suppression, fat absorption, increased metabolism, appropriate nutrition and regular exercise. Studies have been conducted on drugs which block the biosynthesis of TAG by inhibiting relevant enzyme activities (Tomoda et al., 2007).

In plants, TAG is the major component of vegetable oils which are primarily used as cooking oils but can also be used as a renewable feedstock for industrial applications. Plants can be modified by metabolic engineering to serve as green factories for the production of novel industrial materials, nutritionally enhanced foods or pharmaceuticals. For example, vegetable oils can substitute for petroleum in the production of environmentally friendly industrial fluids and lubricants (Metzger et al., 2006); serve as an alternative source of polyunsaturated fatty acids (Truksa et al., 2006); or be converted to biodiesel (Vasudevan et al., 2008). Since the capacity of oilseeds to accumulate oil is significant, several strategies to increase TAG content in seeds have been explored (Weselake, 2002).

Certain industrial applications require plant oils containing fatty acids with specific double bond configuration or functional groups (epoxy, hydroxy) (Jaworski et at, 2003). Many of these fatty acids can be found in plants, but usually in species with limited agronomic potential (Badami et al., 1981). While the key genes involved in the synthesis of unusual fatty acids (e.g. FAD2 desaturases and thioesterases) have been transferred into established crops, the resulting transgenic plants accumulated only modest proportions of novel fatty acids, possibly due to their inefficient incorporation into TAG (Cahoon et al., 1999).

It has been demonstrated that organisms producing high amounts of unusual fatty acids contain TAG synthases which are able to scavenge the unusual fatty acids into TAG (Yu et al., 2006). Specialized TAG synthases which prefer or do not discriminate against novel fatty acids could have a positive effect on the accumulation of unusual fatty acids in crop seed oils by creating a metabolic pull, thereby increasing the efficiency of preceding steps (Cahoon et al., 2007).

The current methods to evaluate neutral lipid synthase enzyme activities require a high degree of proficiency, extensive labour and time, and expensive, hazardous reagents, particularly radio-labelled substrates (Coleman, 1992). There is thus a need for more rapid, efficacious methods which mitigate these disadvantages of the prior art.

### SUMMARY OF THE INVENTION

The present invention relates to high throughput methods of identifying neutral lipid synthases, comprising the steps of positively selecting eukaryotic cells for recombinant neutral lipid synthases. The enzyme activities of the recombinant neutral lipid synthases may then be quantified, such as by using a fluorescence in situ assay, for example. In one embodiment, the cells comprise yeast cells.

In one aspect, the invention comprises a method for identifying a neutral lipid synthase comprising the steps of positively selecting yeast cells impaired of neutral lipid biosynthesis for a neutral lipid synthase by introducing into the yeast cells a vector which expresses the neutral lipid synthase; and culturing the yeast cells under selective conditions thereby positively selecting for cells transfected with the vector.

In one embodiment, the method further comprises the step of quantifying enzyme activity of the recombinant neutral lipid synthase. The enzyme activities of the neutral lipid synthases may be quantified by contacting the yeast cells with a fluorescent dye, wherein the dye interacts with neutral lipids in the yeast cells produced by the neutral lipid synthase.

In one embodiment, the method further comprises the step of isolating the yeast cells with increased fluorescence due to their neutral lipid content using fluorescent-activated cell sorting.

In one embodiment, the positive selection method may be used to isolate or identify preference or non-discrimination against a specific fatty acid or acyl chain by a neutral lipid synthase, comprising the steps of growing transformed yeast cells on growth media supplemented by the specific fatty acid or acyl chain, and measuring levels of neutral lipid production.

In one embodiment, the positive selection method may be used to identify a modulator of a neutral lipid synthase, comprising the steps of co-expressing a candidate modulator in the yeast cells, or growing the yeast cells on growth media comprising a candidate modulator, and measuring levels of neutral lipid production. The candidate modulator may be an inhibitor or a positive modulator of a neutral lipid synthase.

In one embodiment, the yeast cells are of the species Saccharomyces cerevisiae. In one embodiment, the yeast cells are of a knock-out S. cerevisiae strain. In one embodiment, the yeast cells are of a quadruple knock-out S. cerevisiae strain. In one embodiment, the S. cerevisiae strain is quadruple knock-out dga1, iro1, are1 and are2.

Additional aspects and features of the present invention will be apparent in view of the description, which follows. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The invention will now be described by way of an exemplary embodiment with reference to the accompanying simplified, diagrammatic, not-to-scale drawings:

FIG. 1 shows cultures of S. cerevisiae strain H1246 (right column) and the corresponding parental strain (left column) transformed with pYESLacZ or pYESBnDGAT1 were inoculated in YNBG at a final OD600 of 0.1. Oleic acid, dissolved in ethanol at 0.5M, was supplemented to the cultures at the final concentrations indicated. The cultures were incubated at 30 oC, 250 rpm and the growth was monitored for 72 hours. Cultures expressing LacZ or BnDGAT1 are denoted in circles or triangles, respectively.

FIG. 2 shows the results of H1246 yeast strain expressing LacZ (L) or BnDGAT1 (B) inoculated on the corresponding YNBG solid medium and incubated at 30° C. for 6 days. (A) Plates of YNBG with and without supplement of 1 mM of oleic acid (C_{18:1}) dissolved in ethanol. The plate without FA contained the same volume of ethanol only. (B) Plates of YNBG supplemented with 1 mM of palmitoleic (16:1^{cisΔ9}), linoleic (18:2^{cisΔ9,12}), α-linolenic (C_{18:3}), docosahexaenoic (C_{22:6}), ricinoleic (C_{18:1} OH), erucic (C_{22:1}) and 0.5 mM of palmitic (C_{16:0}) and stearic (C_{18:0}) acids. The FAs were dissolved in ethanol at 0.5M or 0.25M and added to the YNBG prior to plating. (C) Plates supplemented with oleic, α-linolenic and docosahexaenoic acids at the final concentrations indicated. (D) Selection of yeast cells in medium with FA after transformation. H1246 cells were transformed with 1 μg of pYESBnDGAT1 (column 1), 1 μg of pYESLacZ (column 2) and 0.1 μg of pYESBnDGAT1 mixed with 0.9 μg of pYESLacZ (column 3). After transformation, yeast cells were recovered in liquid YNBD medium for 6 hours, inoculated in YNBG plates with or without supplement of 1 mM oleic acid and incubated at 30° C. for 6 days.

FIG. 3 shows the characterization of factors influencing NRA. (A) Optimization of Nile red concentration. NRA was performed with 95 μL of H1246 cultures expressing LacZ (dash lines) or BnDGAT1 (full lines) at stationary phase and diluted at different cell densities as described. After measuring the background fluorescence, 5 μL of methanolic solution of Nile red, at different concentrations, were added and followed by the second measurement with 5-minute interval from the first measurement. The difference between the first and second measurement is denoted in Y axis as ΔF in arbitrary units (a.u.) and the final concentration of Nile red in the culture is denoted in the X axis. (B) NRA of the same cultures at stationary phase plotted as a function of cell density (OD 600). Full lines denote linear regression with dashes corresponding to intervals of 99% confidence. (C) NRA performed with mixtures of BnDGAT1- and LacZ-expressing cultures normalized to the same cell density. The full line represents the linear regression with the error bars representing standard deviation.

FIG. 4 shows the validation of the selection system and NRA with mutants of RcDGAT1. (A) NRA and DGAT microsomal activity. Enzyme activity was determined by radioactive assay for each RcDGAT1 variant and NRA results were expressed as ΔF (a.u.) divided by OD600. The table below indicates the selection system results for H1246 cultures expressing RcDGAT1 and the respective variants. Negative (−) and positive (+) indicate the ability to produce colonies in solid YNBG supplemented with 1 mM oleic acid. (B) Relationship between ΔF/OD and the specific activity measured by radioactive assay. The line denotes linear regression; error bars represent standard deviation.

FIG. 5 shows screening of BnDGAT1 mutagenized libraries. Yeast cells expressing mutagenized BnDGAT1 and controls (LacZ- and wild type BnDGAT1-expressing cells) were analyzed through the Nile red fluorescence assay. The numbers in brackets indicate the average values for each group and “n” denotes the number of individual clones tested for each group.

FIG. 6 shows histogram representation of large scale HTS screening. (A) 1528 clones of library A and (B) 200 individual clones of wild type BnDGAT1 were analyzed through the HTS. ΔF/OD values were calculated and distributed through a histogram using a bin width of 80. Gaussian curves, represented by lines, were calculated for each histogram. Normality test applied for the histograms of library A and wild type BnDGAT1 resulted in significance levels of P=<0.0001 and P=0.615, respectively.

FIG. 7 shows an analysis of selected clones of library A. The clones corresponding to ΔF/OD values ranging 0.56 to 0.7 (High) and 0.1 (Low) were individually grown in test tubes until reaching the stationary growth phase and analyzed through the Nile red assay. The numbers in brackets indicate the average values for each group.

### DETAILED DESCRIPTION OF PREFERRED EMBODIMENTS

The present invention provides for high throughput methods of identifying neutral lipid synthases. As will be apparent to those skilled in the art, various modifications, adaptations and variations of the foregoing specific disclosure can be made without departing from the scope of the invention claimed herein. The various features and elements of the described invention may be combined in a manner different from the combinations described or claimed herein, without departing from the scope of the invention.

In one embodiment, the invention comprises a method including the steps of positively selecting yeast cells expressing recombinant neutral lipid synthases, and quantifying the enzyme activities of the recombinant neutral lipid synthases using a fluorescence in situ assay.

In one embodiment, the neutral lipid synthase may be a TAG synthase, an SE synthase, or a wax ester synthase. In specific embodiments, the neutral lipid synthase may comprise one or more of diacylglycerol acyltransferase 1 (DGAT1), diacylglycerol acyltransferase 2 (DGAT2) phospholipid-diacylglycerol acyltransferase (PDAT), acyl-CoA:cholesterol acyltransferase (ACAT), and lecithin:cholesterol acyltransferase (LCAT).

Embodiments of the invention use knock-out strains of a eukaryotic cell, defined herein as a cell having no or substantially reduced background neutral lipid synthase activity. Such knock-out strains may be the result of interrupted genes known to be involved in neutral lipid synthase activity. The eukaryotic cell may comprise a yeast cell, a plant cell, or a mammalian cell.

In the fission yeast Schizosaccharomyces pombe, interruption of dga1 and plh1 (encoding DGAT and PDAT, respectively) leads to lack of TAG biosynthesis and limited viability as cells undergo apoptosis during the stationary growth phase (Zhang et al., 2003). This effect is enhanced by supplementing the growth medium with diacylglycerol or fatty acids. The budding yeast Saccharomyces cerevisiae lacking TAG synthase activity (quadruple knockout DGA1, LRO1, ARE1 and ARE2) is viable under normal growth conditions despite the lack of neutral lipid production (Sandager et al., 2002), but exhibits reduced growth rates compared to wild type on growth medium supplemented with diacylglycerol or fatty acids.

In one embodiment, a knock-out yeast strain is used in a positive selection system for genes conferring neutral lipid synthase activity. In one embodiment, the knock-out strain is a S. cerevisiae strain. In one embodiment, the strain is a quadruple knock-out S. cerevisiae strain. In one embodiment, the S. cerevisiae strain is quadruple knock-out dga1, lro1, are1 and are2. The knock-out strains are less viable, have significantly extended lag growth phase or grow more slowly, in growth media supplemented with DAG or fatty acids, unless they have incorporated a gene which confers neutral lipid synthase activity. Therefore, the cells which have neutral lipid synthase activity will grow significantly faster, allowing their apparent positive selection. In one embodiment, the growth media may be supplemented with a fatty acid such as oleic acid, in concentrations from about 25 μM to about 1000 μM.

In one embodiment, the isolation or selection step may be followed by quantification of the enzyme activity. Neutral lipid synthase activity can be accurately quantified in assays using radio-labelled substrates, with the specific activity of the enzyme being directly proportional to the incorporation of the radioactive label into neutral lipid (Coleman, 1992). The product of the enzymatic reaction may be resolved by thin layer chromatography analysis. Improvements of the DGAT assay have made such an assay more amenable to high throughput screening, alleviating the need for the TLC separation, but still relying on radioactive substrates (Landro et al., 2006; Ramharack et al., 2003).

A method to estimate lipid content of oleaginous microorganisms based on a fluorescent dye, Nile Red, has been reported (Kimura et al., 2004). Nile Red stains most lipids, particularly neutral lipids such as TAG and SE, partly due to the fact that the fluorescence intensity is much higher for neutral lipids than for polar lipids. The maximum wavelength emission of Nile Red conjugated with neutral lipids is different from the maximum of the dye-polar lipid complex (Greenspan et al., 1985). Therefore, activity levels of neutral lipid synthases may be quantified by measuring the fluorescence of cells stained with Nile Red.

The positive selection method described herein may be useful for a variety of applications including, for example, discovery of new neutral lipid synthases with enhanced properties based on the screening of natural (cDNA) or artificial (molecular, directed or in vitro evolution) DNA libraries; screening of potential neutral lipid synthase inhibitors or anti-obesity drugs; screening for stimulators of neutral lipid synthases; use as a routine laboratory assay; or manipulation of the quality and content of vegetable oils.

In vitro evolution of neutral lipid synthases to enhance enzymatic activity and modify substrate selectivity may be performed by combining described assays. In one embodiment, cDNA libraries of a randomly mutagenized neutral lipid synthase may be created using standard techniques (see for example, Stemmer, 1994). Such libraries may then be transformed into yeast cells impaired of neutral lipid biosynthesis which are then screened using the positive selection system described herein. This step eliminates mutated variants of the gene which do not encode proteins with neutral lipid synthase activity. Selected yeast colonies may be then grown in a small volume of liquid medium and used directly to measure the activity of each individual neutral lipid synthase mutant, by a fluorescence assay, for example. Yeast cultures presenting higher fluorescence values contain a neutral lipid synthase variant with enhanced activity. Genes corresponding to these neutral lipid synthases are then subjected to additional cycles of mutagenesis to further increase their enzyme activity. Alternatively, the selection of mutated libraries may be performed by application of Fluorescent-Activated Cell Sorting (FACS).

Current methods to isolate a TAG synthase cDNA or a gene encoding a TAG synthase rely on DNA homology using PCR and DNA hybridization, which are reliable techniques on condition that homologous cDNAs have been previously characterized. For example, in type-1 DGAT, there are several conserved regions that can be used to isolate homologous genes from different organisms (Cases et al., 1998; He et al., 2004; Milcamps et al., 2005; Nykiforuk et al., 2002; Wang et al., 2006; Yu et al., 2006; Zou et al., 1999). However, in type-2 DGAT, the cDNA sequences available in the literature are variable, making homology-based cloning problematic. DGAT3 was recently identified in peanuts (Saha et al., 2006). To date, no homologs of DGAT3 have been found in other organisms.

With regard to other TAG synthases (for example, PDAT), few homologous genes have been cloned and the only functional enzymes have been characterized in yeast and Arabidopsis thaliana (Stahl et al., 2004; Oelkers et al., 2000; Dahlqvist et al., 2000). Certain TAG synthases (for example, diacylglycerol:diacylglycerol transacylase) have been characterized only at the level of enzyme activity with no information yet available pertaining to protein or DNA sequences (Lehner et al., 1993; Stobart et al., 1986).

In one embodiment, methods of the invention may be used to isolate genes encoding neutral lipid synthases, especially from organisms which produce oils with high contents of desirable fatty acids. A cDNA library from such organisms may be constructed in a yeast-expression vector and expressed in the described quadruple knock-out yeast strain. The cells containing an active neutral lipid synthase are then selected on the medium supplemented with fatty acids. The gene of interest is identified by isolating and sequencing the vector from a positively selected colony. To eliminate false-positive clones, the selected colonies are rescreened by measuring their ability to synthesize neutral lipids, such as by the Nile Red fluorescence assay, for example. Yeast cultures with higher fluorescence contain neutral lipid synthases.

Selection and fluorescent assay systems can be used to isolate or identify neutral lipid synthase genes which prefer or do not discriminate against acyl-CoA substrates containing unusual acyl chains such as, for example, polyunsaturated or hydroxylated fatty acid. Such methods can be used to screen natural cDNA libraries prepared from organisms of interest (e.g., very-long-chain polyunsaturated fatty acids-producing marine microorganisms or plant seeds accumulating high proportion of unusual fatty acids such as castor bean). Alternatively, the screening can be performed on populations of mutagenized neutral lipid synthase genes in order to select variants with increased activity with the acyl chain of interest in the process of molecular evolution. Selection is performed by incorporating the free fatty acid of interest in the solid medium or by growing pre-selected yeast cells in the liquid medium containing the fatty acid and measuring the accumulation of neutral lipids by the fluorescent assay described herein.

One embodiment of the present invention can be used to detect and characterize inhibitors of neutral lipid synthases. Excessive accumulation of TAG and SE in certain tissues leads to hypertriglyceridemia, obesity or type-2 diabetes (Rudel et al., 2001; Lehner et al., 1996). The control of neutral lipid biosynthesis can be used as a strategy to treat or prevent such diseases. Several inhibitors of neutral lipid synthases have been reported (Tomoda et al., 2007). Inhibition of TAG biosynthesis has direct impact on fat deposition in muscle and adipocytes, while inhibition of SE formation would decrease development of atherosclerotic lesions either by decreasing formation of macrophage foam cells or by reducing plasma levels of lipoproteins containing ApoB (such as LDL) through a decrease in hepatic and intestinal SE formation.

Current methods to characterize the inhibition of neutral lipid accumulation involve the analysis of lipids produced in mammalian cells (such as rat liver cells and macrophages) cultivated in the presence of the compound of interest (Mayorek et al., 1985; Namatame et al., 1999; Nishikawa et al., 1990). More accurate assays involve the isolation of liver cell microsomes and enzyme assays with radio-labelled substrates (Coleman, 1992) in the presence of the inhibitor (Chung et al., 2004; Lee et al., 2006; Chung et al., 2006).

It will be appreciated that the screening for inhibitors may involve two different strategies. If the potential modulators of neutral lipid synthesis are single gene products, such as proteins or peptides, the yeast cells can be co-transformed with a library encoding a natural or combinatorial population of such products besides the gene for a neutral lipid synthase of interest. Alternatively, the potential inhibitors can be delivered exogenously by growing the yeast cultures in their presence.

In one embodiment, a yeast strain impaired of neutral lipid biosynthesis may be transformed with a cDNA encoding a mammalian neutral lipid synthase. Upon appropriate induction of the cDNA expression, the cell strain will produce neutral lipids (such as TAG or SE), which may be measured, such as by the Nile Red in situ assay. However, when cells are grown in the presence of a neutral lipid synthase inhibitor, the reduction in the biosynthesis of neutral lipid will be reflected in lower fluorescence signal.

Advantageously, this assay can be performed in higher throughput (for example using 96 multi-well plates or FACS) at lower cost and effort compared to prior art methods. In addition, the method facilitates screening and selection of specific inhibitors of single polypeptides with neutral lipid synthase activity. This is desirable from the pharmacology perspective, since broad-spectrum inhibitors have higher probability to cause adverse effects. Examples of such adverse effects have been observed for inhibitors of SE synthase. The last step of SE biosynthesis in mammals is catalyzed by ACAT and there are two isoforms of ACAT in humans (ACAT1 and ACAT2), each presenting distinct expression pattern across the tissues (Lee et al., 2000). ACAT2 is predominately expressed in the liver and to a lesser extent in the small intestine, while ACAT1 is ubiquitously expressed in most other tissues (Parini et al., 2004; Buhman et al., 2000). Several inhibitors of ACAT have been reported, with at least two having been tested in humans without success (Tomoda et al., 2007; Fazio et al., 2006). These drugs, namely avasimibe and pactimibe, are nonselective ACAT inhibitors and have been proven ineffective against atherosclerosis and probably harmful due to ACAT1 inhibition (Tardif et al., 2004; Nissen et al., 2006). The selectivity of ACAT inhibitors has not been well studied with the exception of pyripyropene (Ohshiro et al., 2007). However, specific inhibition of ACAT2 via antisense oligonucleotides in mice decreases diet-induced hypercholesterolemia and severely reduces SE deposition in arteries (Bell et al., 2006). Decreased levels of saturated and monounsaturated fatty acids in SE in plasma LDL and increased levels of polyunsaturated fatty acids were also reported, indicating that specific inhibition of ACAT2 is a feasible and promising strategy to treat or prevent atherosclerosis (Farese, 2006).

A similar scenario is found in mammalian TAG biosynthesis, although no clinical trials have been yet reported. TAG is mainly synthesized by the two isoforms of DGAT (DGAT1 and DGAT2). Studies using mice knock-outs revealed that DGAT1 deficiency protects against insulin resistance and diet-induced obesity (Smith et al., 2000; Chen et al., 2002). However, DGAT2 knockout mice are not viable, dying shortly after birth (Stone et al., 2004). Although no drug to inhibit DGAT has yet been developed, considering the results with mice knock-outs, it was hypothesized that the reduction of DGAT2 activity might result in undesirable effects (Tomoda et al., 2007). It is thus important that potential DGAT inhibitors for potential drug development are strictly specific to one type of DGAT.

The same principle used to identify inhibitors of neutral lipid synthases may be applied in the identification of positive modulators of neutral lipid synthases. Such regulators would be useful to increase storage lipid synthesis in oilseeds or oleaginous microorganisms through metabolic engineering.

Embodiments of the present invention provides numerous practical advantages over methods of the prior art which presents time-consuming, expensive technologies. Since the invention incorporates a yeast strain which is substantially devoid of background neutral lipid synthase activity, any neutral lipid which accumulates in the yeast cells is directly attributable to the activity of the recombinant neutral lipid synthase. Further, in one embodiment, the invention eliminates the need for expensive radio-labelled substrates. In one embodiment, the invention may be performed in situ, thus overcoming the need for sample preparation.

The invention can be incorporated with other analyses such as, for example, high throughput screening which requires analysis of a large number of individual samples arrayed in a large multi-well plate, such as 96-well or 384-well plates well known to those skilled in the art. Such a combined system facilitates the screening of many individual recombinant polypeptides for neutral lipid synthase activity, and the evaluation of the effects of compounds modulating the activity of a single polypeptide on a mass scale.

The fluorescent assay for neutral lipid synthase activity can be combined with fluorescent cell sorting (FACS) to increase the efficiency of selection and the throughput (approximately one million individual cells per hour). The methods described herein may be used either individually or in combination to identify or isolate TAG synthase enzymes with enhanced or specialized activity.

The Examples provided below are not intended to be limited to these examples alone, but are intended only to illustrate and describe the invention rather than limit the claims that follow.

### EXAMPLES

### Example 1

### Positive Selection

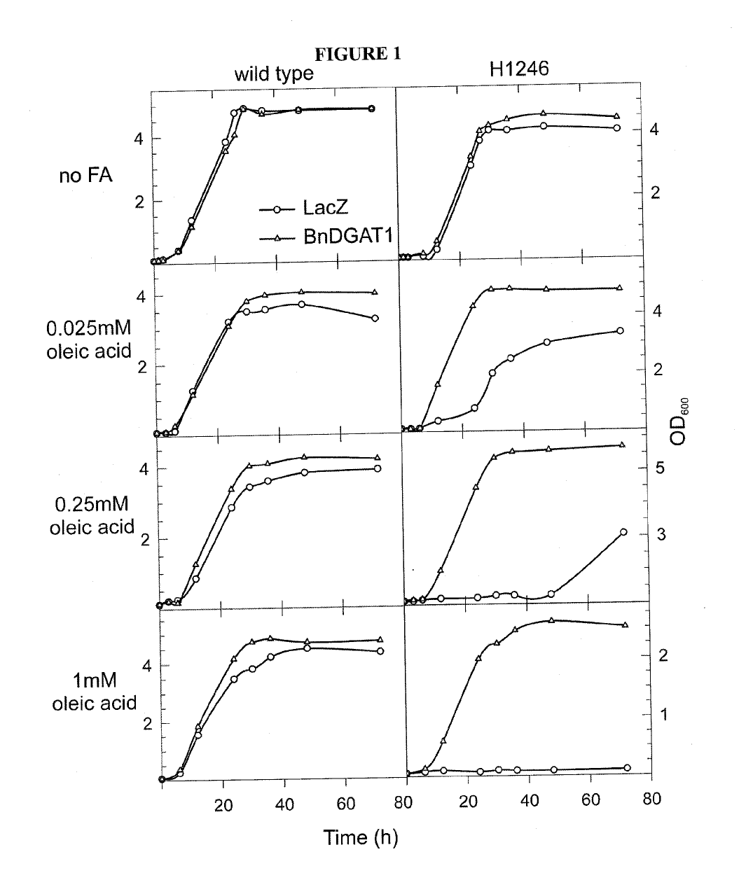

Three yeast strains (wild type, dga1 knock-out and quadruple knock-out) were transformed with yeast expression vector pYES2.1-TOPO (Invitrogen) containing a cDNA coding for DGAT1 from several oilseed plants (canola, flax or castor bean). The same vector containing the gene coding for the bacterial protein LacZ served as the negative control. Transformed yeast cells were cultivated in 50 mL of uracil drop-out medium supplemented with 2% glucose for 48 hours shaking at 30° C. and 250 rpm. The cells were washed twice with water and inoculated in liquid media supplemented with 2% galactose, 1% raffinose to induce the expression of the recombinant proteins, and different concentrations of free fatty acids (0 to 1000 μM of oleic acid). Free fatty acids from the medium can be imported by yeast cells and immediately converted to their acyl-CoA equivalents, thus becoming substrates for TAG synthases (Faergeman et al., 2001). Cell growth was measured for a period of 72 hours. In the media containing fatty acids, knock-out strains had to express a recombinant DGAT1 to achieve growth rates comparable to that of the wild type yeast. The inhibitory effect of oleic acid was observed at a concentration as low as 25 μM, but 1000 μM concentration of fatty acid was the most effective in distinguishing the strains with and without TAG synthase activity (FIG. 1).

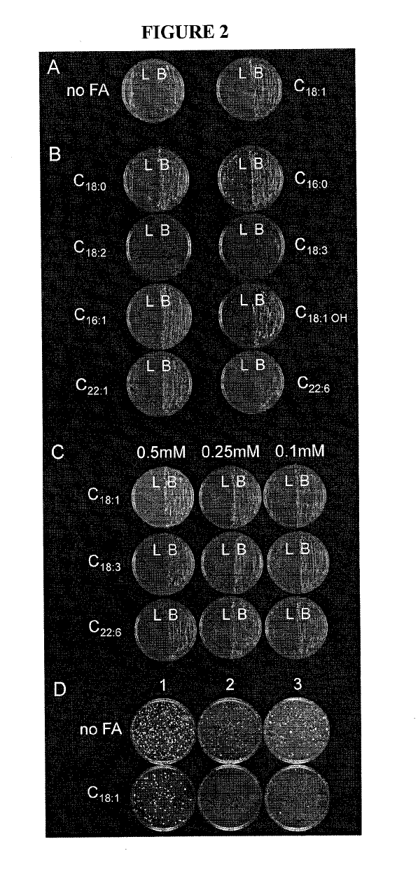

The positive selection of yeast cells possessing the TAG synthase activity is also reproducible on a solid medium. The quadruple knock-out strain cultures harboring vector with either DGAT1 or LacZ gene were plated onto agar-solidified uracil drop-out medium supplemented with 2% galactose, 1% raffinose and 1000 μM oleic acid. After five days of incubation at 30° C., only the cells expressing the recombinant DGAT1 formed visible colonies. The TAG synthase activity of these colonies was confirmed by an independent enzyme assay. The exposure of yeast S. cerevisiae cells to the growth medium containing fatty acids positively selects for the cells possessing the TAG synthase (DGAT in an exemplary example) activity (FIG. 2A).

The positive selection can be obtained with several different fatty acids. The quadruple knock-out strain cultures harboring vector with either DGAT1 or LacZ gene were plated onto agar-solidified uracil drop-out medium supplemented with 2% galactose, 1% raffinose and a range of fatty acids differing in the carbon-chain length as well as in the degree of saturation. The growth of the control strain was inhibited in most cases except when palmitic, stearic and erucic acids were supplemented, most likely due to their lower dispersion in the aqueous medium. Lowering the concentration of these FAs to 0.5 mM seemed to help their dispersion in the medium but it did not substantially improve the selectivity of the media (FIG. 2B). Supplement of 1 mM linoleic, α-linolenic or docosahexaenoic acids, on the other hand, inhibited the growth of both cultures. Supplement of these fatty acids at a range of lower concentrations indicated that 500 μM concentration of fatty acid 0.5 mM concentrations of linoleic, α-linolenic or docosahexaenoic acids were suitable for selection (FIG. 2C).

### Example 2

### Nile Red Fluorescence Assay

A volume of 95 μL of yeast culture is placed in a well of a 96-well plate and the background fluorescence is measured using a 96-well plate fluorimeter (Fluoroskan Ascent™ Thermo) with an excitation filter 485 nm and emission filter of 538 nm. Five microliters of Nile Red solution in methanol (0.8 mg/mL) is then added directly to the yeast cell culture and incubated for five minutes at room temperature. The dye enters the cells and forms fluorescent complexes with neutral lipids. A second fluorescence measurement is performed using the same conditions. The increase in the fluorescence values (ΔF) is directly proportional to the accumulation of neutral lipid in the yeast cells and correlates positively with specific activity of the expressed TAG synthase.

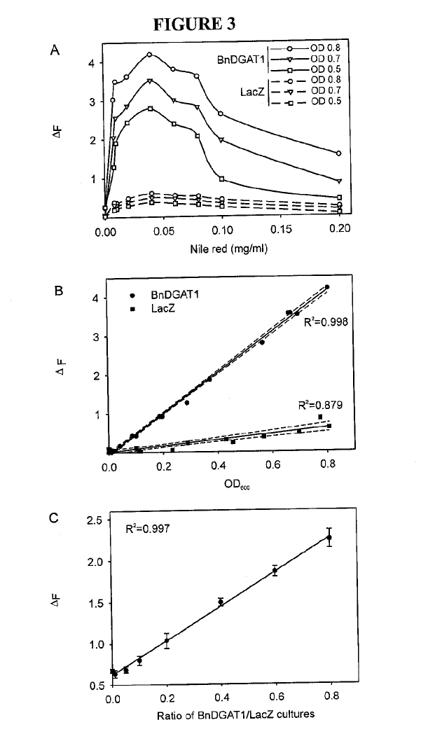

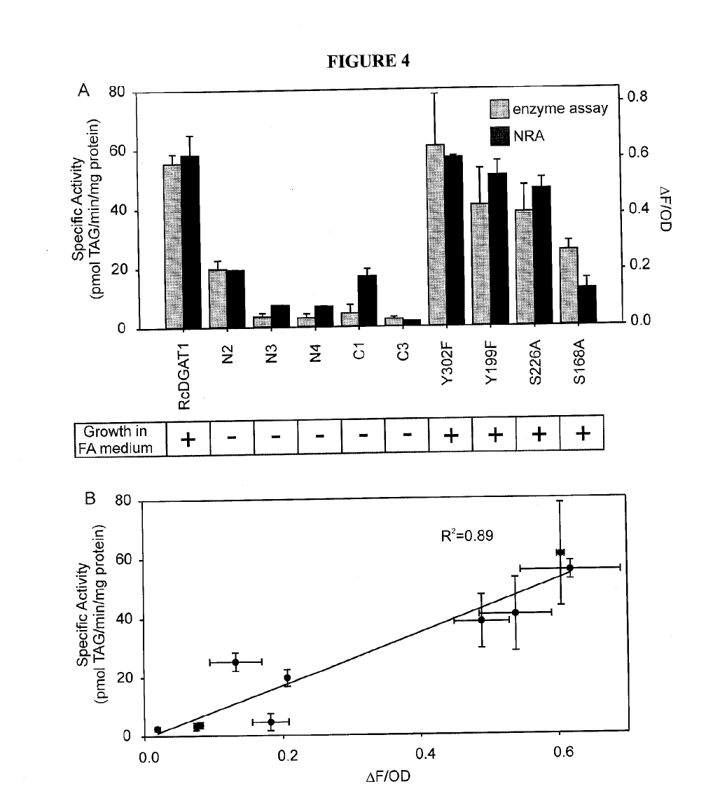

Although 0.8 mg/mL of Nile red methanolic solution gives the highest increase of fluorescence, concentrations up to 0.4 mg/mL can also be used can be used to differentiate between ΔF values obtained for LacZ- and BnDGAT1-expressing cultures (FIG. 3A). The cell density does not affect the concentrations at which maximal ΔF values are observed but it alters the measured fluorescence values. In fact, the cell density obtained by OD₆₀₀ correlates linearly with ΔF values which is not affected by the medium itself (FIG. 3B). Consequently, it is possible to normalize ΔF values by calculating the ΔF/OD ratio rather than trying to achieve the same cell density across samples, which can be impractical with a large number of samples. The efficacy of the Nile red assay in detecting DGAT screening system can be evaluated using mutants of a neutral lipid synthase. Several mutants of a castor bean DGAT1 (RcDGAT1) were constructed by truncation of the N-terminus (N2, N3 and N4), C-terminus (C1 and C3) as well as by the substitution of single residues (Y302F, Y199F, S226A and S168A) through site-directed mutagenesis. These mutants display a wide range of DGAT activity, providing a useful model for validation of the novel methods. RcDGAT1-expressing cells displayed normal growth on medium supplemented with 1 mM oleic acid. The mutants Y302F, Y199F, S226A and S168A also grew normally while no growth could be detected for N2, N3, N4, C1 and C3 over the same period of incubation. Nile red assay and the radioactive in vitro assay with liquid cultures expressing RcDGAT1 variants were also performed. Briefly, the relative comparison of DGAT activity of the wild type and the modified RcDGAT1 variants measured by NRA resembled the results of the in vitro enzyme assay. A positive correlation was found between the Nile red and the conventional in vitro enzyme assay (FIG. 4).

### Example 3

### Molecular Evolution of TAG Synthases

Mutagenesis by epPCR introduces random variations in the amplified coding sequence. Besides the substitutions introducing stop codons that result in truncated polypeptides, it is predicted that a large proportion of amino acid modifications will be detrimental to enzyme activity and only few mutations can increase the enzyme activity. To eliminate inactive variants and narrow down the scope of subsequent experiments to only clones expressing active DGAT variants the clones can be selected as demonstrated in the first example. A cDNA encoding DGAT1 from Brassica napus, was used as a template in the construction of mutagenized libraries. Libraries of randomly mutagenized BnDGAT1 were generated by error-prone PCR (epPCR). Three different reaction conditions leading to progressively increasing mutation rates were used to generate populations of mutagenized cDNAs. The populations where denoted libraries A, B and C with 1.5, 2.2 and 3.8 estimated mean number of amino acid substitutions per variant, respectively. Positive selection of these libraries indicated that the number of colonies formed on the FA selection medium was inversely proportional to the mean mutation rate of the library. The reduction in the number of growing colonies under the selective conditions suggests that a large proportion of introduced amino acid substitutions had a negative effect on DGAT activity. This observation further underscores the requirement for the positive selection system.

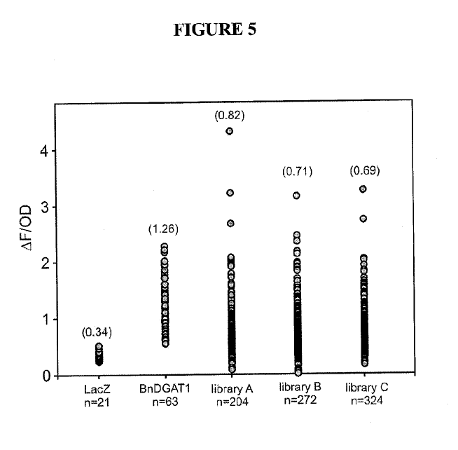

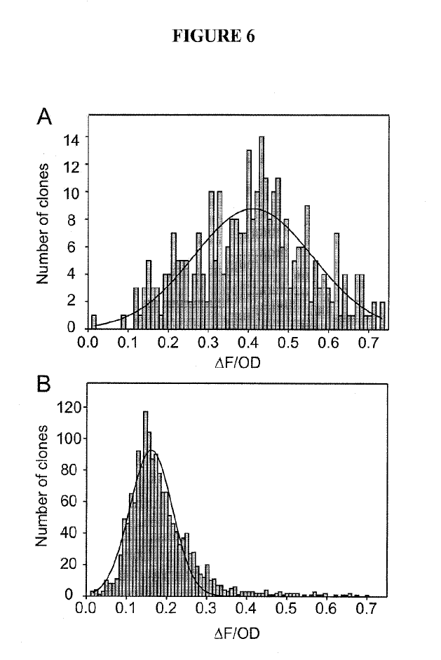

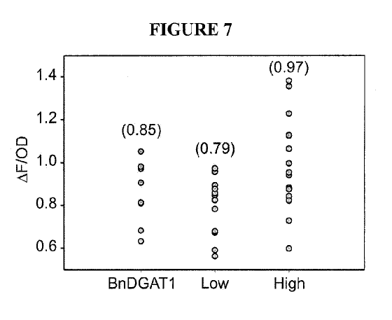

After selecting yeast clones expressing active variants of BnDGAT1, screening of libraries A, B and C to characterize TAG accumulation in cells expressing BnDGAT1 mutants was performed. A sample of about 200 to 300 colonies from each library was cultivated in 96-well plates and evaluated using the Nile red fluorescence assay. Yeast colonies transformed with pYESLacZ and pYESBnDGAT1 were used as controls. The mean as well as the range of ΔF/OD values for cultures expressing LacZ or BnDGAT1 was clearly different, while the means for mutagenized libraries were between the two controls (FIG. 5). The distribution analysis indicated that only ΔF/OD values for LacZ- and BnDGAT1-expressing cells followed a normal distribution. Library A resulted in the highest ΔF/OD mean and a larger screening of this set was performed. In this larger experiment 1528 clones from library A were compared to the reference of 200 individual clones of pYESBnDGAT1. Similar to the previous experiment, the mean of ΔF/OD values for library A was lower (0.19) compared to the mean for the BnDGAT1-expressing cells (0.4). Furthermore, distribution analysis indicated that only the subset of cells expressing wild type BnDGAT1 passed the normality test, reflecting the intrinsic heterogeneity for the subset of clones comprising library A (FIG. 6). The normal distribution determined for BnDGAT1-expressing cells was mainly a result of technical variability. The observed range of ΔF/OD values were similar for both sets, but, considering the distribution caused by technical variation, it is possible that some of the BnDGAT1 variants represented by individual clones could be more active than the wild type. To verify the reproducibility of the observed values two batches of clones were selected from library A based on their fluorescence values: “High” with ΔF/OD values ranging 0.56 to 0.7 and “Low” with ΔF/OD of 0.1. These cultures, together with reference clones of BnDGAT1 were individually grown in larger volume of liquid YNBG and analyzed again by Nile red fluorescence assay. The spread between the ΔF/OD means indicated that the differences in fluorescent values were transferred to the secondary cultures and are most likely caused by genetic modifications of BnDGAT1 (FIG. 7).

### Example 4

### Isolation of TAG Synthase cDNAs from Natural cDNA Libraries

A controlled DNA blend is used to isolate TAG synthase cDNA. In three individual experiments, the quadruple knock-out yeast strain was transformed with equal amounts of the following plasmids: pYES-LacZ (negative control), pYES-BnDGAT1 (positive control) and a mixture of 90% of pYES-LacZ and 10% of pYES-BnDGAT1. Following transformation, yeast cells were cultivated in the medium supplemented with oleic acid (1 mM) to select for active TAG synthases. The experiment containing the mixture of plasmids (90% negative and 10% positive) resulted in a number of actively growing colonies which represented 10.1% of colonies in the experiment consisting of 100% positive control (pYES-BnDGAT1) (FIG. 2D). The close relationship between the number of colonies selected and the relative representation of the positive control in the vector mix indicates that TAG synthases may be isolated from complex mixtures of cDNA-carrying expression vectors, such as, for example, libraries of organisms producing unusual fatty acids. If, for example, a TAG synthase is represented 1.0×10⁻⁵ in a natural cDNA library, it would be necessary to screen 1.0×10⁶ yeast colonies to have 90% probability to isolate the desired cDNA. Considering the efficiency of yeast transformation of 2.0×10⁵/1 μg DNA/10⁸ cells, it will only be necessary to use 5 μg of a cDNA-library vector for one screening experiment, which is a reasonable amount.

### Example 5

### Selection of TAG Synthase Genes With Higher Selectivity to Certain Fatty Acids

Yeast cultures expressing BnDGAT1 and RcDGAT1 genes from B. napus and Ricinus communis (castor bean) respectively, were grown in liquid media containing erucic or ricinoleic acid. The culture expressing the BnDGAT1 accumulated more neutral lipids in the medium containing erucic acid, which is naturally present in Brassica seed oil, than in the medium with ricinoleic acid. In contrast, in the medium with ricinoleic acid (a fatty acid which represents a large proportion of the castor bean oil), yeast expressing RcDGAT1 accumulated more neutral lipids than the BnDGAT1-expressing culture.

### Example 6

### Screening and Characterization of Inhibitors of Neutral Lipid Metabolism

A yeast strain devoid of neutral lipid synthesis is transformed with a cDNA encoding a mammalian TAG or SE synthase. Upon appropriate induction of the cDNA expression, the cell strain produces neutral lipids (TAG or SE), resulting in high fluorescence increase in the Nile Red in situ assay. However, when cells are grown in the presence of a TAG or SE synthase inhibitor, the reduction in the biosynthesis of neutral lipid will be reflected in lower fluorescence signal.

### Example 7

### Screening and Isolation of Novel Specific Modulators of TAG Synthase

A yeast strain devoid of neutral lipid biosynthesis is transformed with a cDNA encoding a TAG synthase. The modulator is delivered exogenously in the medium or produced internally (in the case of proteins and peptides) through co-transformation of the cells with DNA libraries. Upon appropriate induction of the recombinant gene expression, the cell strain produces TAG, resulting in a certain level of fluorescence in the Nile Red in situ assay. Upon positive induction of the TAG activity caused by the presence of the interacting compound, the level of fluorescence in the cell will increase. With regard to internally produced modulators, the throughput of screening can be increased by employing FACS technology.

### REFERENCES

All publications mentioned in this specification are indicative of the level of skill of those skilled in the art to which this invention pertains. Where permitted, all publications are herein incorporated by reference to the same extent as if each individual publication was specifically and individually indicated to be incorporated by reference.

Badami, R. C. and Patil, K. B. (1981) Structure and occurrence of unusual fatty acids in minor seed oils. Prog. Lipid Res. 19:119-153.

Bell, T. A., Brown, M., Graham, M. J., Lemonidis, K. A., Crooke, M. and Rudel, L. L. (2006) Liver-specific inhibition of acyl-coenzyme A: cholesterol acyltransferase 2 with antisense oligonucleotides limits atherosclerosis development in apolipoprotein B100-only low-density lipoprotein receptor(−/−) mice. Arterioscler. Thromb. Vasc. Biol. 26:1814-1820.

Buhman, K. F., Accad, M., and Farese, R. V. (2000) Mammalian acyl-CoA: cholesterol acyltransferases. Biochim. Biophys. Acta 1529(1-3):142-154.

Cahoon, E. B., Carlson, T. J., Ripp, K. G., Schweiger, B. J., Cook, G. A., Hall, S. E. and Kinney, A. J. (1999) Biosynthetic origin of conjugated double bonds: Production of fatty acid components of high-value drying oils in transgenic soybean embryos. Proc. Natl. Acad. Sci. U.S.A. 96:12935-12940.

Cahoon, E. B., Shockey, J. M., Dietrich, C. R., Gidda, S. K., Mullen, R. T. and Dyer, J. M. (2007) Engineering oilseeds for sustainable production of industrial and nutritional feedstocks: solving bottlenecks in fatty acid flux. Curr. Opin. Plant Biol. 10:236-244.

Cases, S., Smith, S. J., Zheng, Y. W., Myers, H. M., Lear, S. R., Sande, E., Novak, S., Collins, C., Welch, C. B., Lusis, A. J., Erickson, S. K. and Farese, R. V. (1998) Identification of a gene encoding an acyl CoA : diacylglycerol acyltransferase, a key enzyme in triacylglycerol synthesis. Proc. Natl. Acad. Sci. U.S.A. 95:13018-13023.

Chen, H. C. and Farese, R. V. (2005) Inhibition of triglyceride synthesis as a treatment strategy for obesity—Lessons from DGAT1-deficient mice. Arterioscler. Thromb. Vasc. Biol. 25:482-486.

Chen, H. C., Smith, S. J., Ladha, Z., Jensen, D. R., Ferreira, L. D., Pulawa, L. K., McGuire, J. G., Pitas, R. E., Eckel, R. H. and Farese, R. V. (2002) Increased insulin and leptin sensitivity in mice lacking acyl CoA : diacylglycerol acyltransferase 1. J. Clin. Invest. 109:1049-1055.

Chung, M. Y., Rho, M. C., Ko, J. S., Ryu, S. Y., Jeune, K. H., Kim, K. H., Lee, H. S. and Kim, Y. K. (2004) In vitro inhibition of diacylglycerol acyltransferase by prenylflavonoids from Sophora flavescens. Planta Medica 70:258-260.

Chung, M. Y., Rho, M. C., Lee, S. W., Park, H. R., Kim, K., Lee, I. A., Kim, D. H., Jeune, K. H., Lee, H. S., and Kim, Y. K. (2006) Inhibition of diacylglycerol acyltransferase by betulinic acid from Alnus hirsuta. Planta Medica 72:267-269.

Coleman, R. A. (1992) Diacylglycerol acyltransferase and monoacylglycerol acyltransferase from liver and intestine. Methods Enzymol. 209:98-104.

Czabany, T., Athenstaedt, K., and Daum, G. (2007) Synthesis, storage and degradation of neutral lipids in yeast. Biochim. Biophys. Acta 1771:299-309.

Dahlqvist, A., Stahl, U., Lenman, M., Banas, A., Lee, M., Sandager, L., Ronne, H., and Stymne, H. (2000) Phospholipid:diacylglycerol acyltransferase: An enzyme that catalyzes the acyl-CoA-independent formation of triacylglycerol in yeast and plants. Proc. Natl. Acad. Sci. U.S.A. 97:6487-6492.

Dat, N. T., Cai, X. F., Rho, M. C., Lee, H. S., Bae, K., and Kim, Y. H. (2005) The inhibition of diacylglycerol acyltransferase by terpenoids from Youngia koidzumiana. Arc. Pharm. Res. 28:164-168.

Faergeman, N. J., Black, P. N., Zhao, X. D., Knudsen, J. and DiRusso, C. C. (2001) The acyl-CoA synthetases encoded within FAA1 and FAA4 in Saccharomyces cerevisiae function as components of the fatty acid transport system linking import, activation, and intracellular utilization. J. Biol. Chem. 276:37051-37059.

Farese, R. V. (2006) The nine lives of ACAT inhibitors. Arterioscler. Thromb. Vasc. Biol. 26:1684-1686.

Farese, R. V., Cases, S. and Smith, S. J. (2000) Triglyceride synthesis: insights from the cloning of diacylglycerol acyltransferase. Curr. Opin. Lipidol. 11:229-234.

Fazio, S. and Linton, M. (2006) Failure of ACAT inhibition to retard atherosclerosis. N. Engl. J. Med. 354:1307-1309.

Greenspan, P., Mayer, E. P. and Fowler, S. D. (1985) Nile Red—a selective fluorescent stain for intracellular lipid droplets. J. Cell Biol. 100:965-973.

He, X. H., Turner, C., Chen, G. Q., Lin, J. T. and Mckeon, T. A. (2004) Cloning and characterization of a cDNA encoding diacylglycerol acyltransferase from castor bean. Lipids 39:311-318.

Jaworski, J. and Cahoon, E. B. (2003) Industrial oils from transgenic plants. Curr. Opin. Plant Biol. 6:178-184.

Kimura, K., Yamaoka, M. and Kamisaka, Y. (2004) Rapid estimation of lipids in oleaginous fungi and yeasts using Nile red fluorescence. J. Microbiol. Methods 56:331-338.

Landro, J. A., Osterman, D. G. and Pickett, W. Method for assaying enzyme activity. U.S. Pat. No. 6,994,956, issued Feb. 7, 2006.

Lee, R. G., Willingham, M. C., Davis, M. A., Skinner, K. A. and Rudel, L. L. (2000) Differential expression of ACAT1 and ACAT2 among cells within liver, intestine, kidney, and adrenal of nonhuman primates. J. Lipid Res. 41:1991-2001.

Lee, S. W., Rho, M. C., Park, H. R., Choi, J. H., Kang, Y., Lee, J. W., Kim, K., Lee, H. S., and Kim, Y. K. (2006) Inhibition of diacylglycerol acyltransferase by alkamides isolated from the fruits of Piper longum and Piper nigrum J. Agric. Food Chem. 54:9759-9763.

Lehner, R. and Kuksis, A. (1993) Triacylglycerol synthesis by an sn-1,2(2,3)-diacylglycerol transacylase from rat intestinal microsomes. J. Biol. Chem. 268:8781-8786.

Lehner, R. and Kuksis, A. (1996) Biosynthesis of triacylglycerols. Prog. Lipid Res. 35:169-201.

Lung, S. C. and Weselake, R. J. (2006) Diacylglycerol acyltransferase: A key mediator of plant triacylglycerol synthesis. Lipids 41:1073-1088.

Mayorek, N. and Tana, J. B. (1985) Inhibition of diacylglycerol acyltransferase by 2-bromooctanoate in cultured rat hepatocytes. J. Biol. Chem. 260:6528-6532.

Metzger, J. O. and Bomscheuer, U. (2006) Lipids as renewable resources: current state of chemical and biotechnological conversion and diversification. Appl. Microbiol. Biotech. 71 :13-22.

Milcamps, A., Tumaney, A. W., Paddock, T., Pan, D. A., Ohlrogge, J. and Pollard, M. (2005) Isolation of a gene encoding a 1,2-diacylglycerol-sn-acetyl-CoA acetyltransferase from developing seeds of Euonymus alatus. J. Biol. Chem. 280:5370-5377.

Namatame, I., Tomoda, H., Arai, H., Inoue, K. and Omura, S. (1999) Complete inhibition of mouse macrophage-derived foam cell formation by triacsin C. J. Biochem. 125:319-327.

Nishikawa, K., Arai, H., and Inoue, K. (1990) Scavenger receptor-mediated uptake and metabolism of lipid vesicles containing acidic phospholipids by mouse peritoneal-macrophages. J. Biol. Chem. 265:5226-5231.

Nissen, S. E., Tuzcu, E. M., Brewer, H. B., Sipahi, I., Nicholls, S. J., Ganz, P., Schoenhagen, P., Waters, D. D., Pepine, C. J., Crowe, T. D., Davidson, M. H., Deanfield, J. E., Wisniewski, L. M., Hanyok, J. J. and Kassalow, L. M. (2006) Effect of ACAT inhibition on the progression of coronary atherosclerosis. New Engl. J. Med. 354:1253-1263.

Nykiforuk, C. L., Furukawa-Stoffer, T. L., Huff, P. W., Sarna, M., Laroche, A., Moloney, M. M. and Weselake, R. J. (2002) Characterization of cDNAs encoding diacylglycerol acyltransferase from cultures of Brassica napus and sucrose-mediated induction of enzyme biosynthesis. Biochim. Biophys. Acta 1580:95-109.

Oelkers, P., Tinkelenberg, A., Erdeniz, N., Cromley, D., Billheimer, J. T. and Sturley, S. L. (2000) A lecithin cholesterol acyltransferase-like gene mediates diacylglycerol esterification in yeast. J. Biol. Chem. 275:15609-15612.

Ohshiro, T., Rudel, L. L., Omura, S. and Tomoda, H. (2007) Selectivity of microbial acyl-CoA: cholesterol acyltransferase inhibitors toward isozymes. J. Antibiot. 60:43-51.

Parini, P., Davis, M., Lada, A. T., Erickson, S. K., Wright, T. L., Gustafsson, U., Sahlin, S., Einarsson, C., Eriksson, M., Angelin, B., Tomoda, H., Omura, S., Willingham, M. C., and Rudel, L. L. (b 2004) ACAT2 is localized to Hepatocytes and is the major cholesterol-esterifying enzyme in human liver. Circulation 110:2017-2023.

Ramharack, R. R. and Spahr, M. A. Diacylglycerol acyltransferase (DGAT) assay. U.S. Pat. No. 6,607,893, issued Aug. 19, 2003.

Rudel, L. L., Lee, R. G., and Cockman, T. L. (2001) Acyl coenzyme A: cholesterol acyltransferase types 1 and 2: structure and function in atherosclerosis. Curr. Opin. Lipidol. 12:121-127.

Saha, S., Enugutti, B., Rajakumari, S. and Rajasekharan, R. (2006) Cytosolic triacylglycerol biosynthetic pathway in oilseeds. Molecular cloning and expression of peanut cytosolic diacylglycerol acyltransferase. Plant Physiol. 141:1533-1543.

Sandager, L., Gustaysson, M. H., Stahl, U., Dahlqvist, A., Wiberg, E., Banas, A., Lenman, M., Ronne, H. and Stymne, S. (2002) Storage lipid synthesis is non-essential in yeast. J. Biol. Chem. 277:6478-6482.

Siloto, R. M. P., Truksa, M., Brownfield, D., Good, A. G., Weselake, R. J., (2009) Directed evolution of acyl-CoA:diacylglycerol acyltransferase: Development and characterization of Brassica napus DGAT1 mutagenized libraries. Plant Physiol. Biochem.

Smith, S. J., Cases, S., Jensen, D. R., Chen, H. C., Sande, E., Tow, B., Sanan, D. A., Raber, J., Eckel, R. H. and Farese, R. V. (2000) Obesity resistance and multiple mechanisms of triglyceride synthesis in mice lacking Dgat. Nature Genetics 25:87-90.

Stahl, U., Carlsson, A. S., Lenman, M., Dahlqvist, A., Huang, B. Q., Banas, W., Banas, A. and Stymne, S. (2004) Cloning and functional characterization of a phospholipid: diacylglycerol acyltransferase from Arabidopsis. Plant Physiol. 135:1324-1335.

Stemmer, W. P. C. (1994) Rapid evolution of a protein in-vitro by DNA shuffling. Nature 370:389-391.

Stobart, A. K., Stymne, S. and Hoglund, S. (1986) Safflower microsomes catalyze oil accumulation in vitro—a model system. Planta 169:33-37.

Stone, S. J., Myers, H. M., Watkins, S. M., Brown, B. E., Feingold, K. R., Elias, P. M. and Farese, R. V. (2004) Lipopenia and skin barrier abnormalities in DGAT2-deficient mice. J. Biol. Chem. 279:11767-11776.

Tardif, J. C., Gregoire, J., L'Allier, P. L., Anderson, T. J., Bertrand, O., Reeves, F., Title, L. M., Alfonso, F., Schampaert, E., Hassan, A., McLain, R., Pressler, M. L., Ibrahim, R., Lesperance, J., Blue, J., Heinonen, T. and Rodes-Cabau, J. (2004) Effects of the acyl coenzyme A: cholesterol acyltransferase inhibitor avasimibe on human atherosclerotic lesions. Circulation 110:3372-3377.

Tomoda, H. and Omura, S. (2007) Potential therapeutics for obesity and atherosclerosis: Inhibitors of neutral lipid metabolism from microorganisms. Pharmacol. Ther. 115:375-389.

Truksa, M., Wu, G. H., Vrinten, P. and Qiu, X. (2006) Metabolic engineering of plants to produce very long-chain polyunsaturated fatty acids. Transgenic Res. 15:131-137.

Vasudevan, P. T. and Briggs, M. (2008) Biodiesel production-current state of the art and challenges. J. Ind. Microbiol. Biotechnol.

Wang, H. W., Zhang J. S., Gai J. Y. and Chen S. Y. (2006) Cloning and comparative analysis of the gene encoding diacylglycerol acyltransferase from wild type and cultivated soybean. Theor. AppL Genetics: 1-12.

Weselake, R. (2002) Biochemistry and Biotechnology of TAG Accumulation in Plants. In Lipid Biotechnology, T. M. Kuo and H. W. Gardner, eds (Peoria, Ill.: Marcel Dekker), pp. 27-56.

Yu, K. S., McCracken, C. T., Li, R. Z., and Hildebrand, D. F. (2006) Diacylglycerol acyltransferases from Vernonia and Stokesia prefer substrates with vernolic acid. Lipids 41:557-566.

Zhang, Q., Chieu, H. K., Low, C. P., Zhang, S. C., Heng, C. K., and Yang, H. Y. (2003) Schizosaccharomyces pombe cells deficient in triacylglycerols synthesis undergo apoptosis upon entry into the stationary phase. J. Biol. Chem. 278:47145-47155.

Zou, J. T., Wei, Y. D., Jako, C., Kumar, A., Selvaraj, G., and Taylor, D. C. (1999) The Arabidopsis thaliana TAG1 mutant has a mutation in a diacylglycerol acyltransferase gene. Plant J. 19:645-653.

1. A method for identifying a neutral lipid synthase comprising the steps of positively selecting yeast cells for a recombinant neutral lipid synthase by introducing into the yeast cells a vector which expresses a polypeptide for a recombinant neutral lipid synthase; and culturing the yeast cells under selective conditions thereby selecting for cells transfected with the vector.

2. The method of claim 1 further comprising the step of quantifying enzyme activity of the recombinant neutral lipid synthase.

3. The method of claim 1, wherein the yeast cells are cultured on medium supplemented with fatty acids.

4. The method of claim 1, wherein the enzyme activities of the recombinant neutral lipid synthases are quantified by contacting the yeast cells with a fluorescent dye, wherein the dye interacts with neutral lipids in the yeast cells produced by recombinant neutral lipid synthases having enzyme activities.

5. The method of claim 4, further comprising the step of isolating the yeast cells with increased fluorescence due to their neutral lipid content using fluorescent-activated cell sorting.

6. The method of claim 4, wherein the fluorescent dye is Nile Red.

7. The method of claim 1, adapted to isolate or identify preference or non-discrimination against a specific fatty acid or acyl chain by a neutral lipid synthase, comprising the steps of growing transformed knock-out yeast cells on growth media supplemented by the specific fatty acid or acyl chain, and measuring levels of neutral lipid production.

8. The method of claim 1, adapted to identify a modulator of a neutral lipid synthase, comprising the steps of co-expressing a candidate modulator in the yeast cells, or growing the yeast cells on growth media comprising a candidate modulator, and measuring levels of neutral lipid production.

9. The method of claim 8 wherein the candidate modulator is an inhibitor of a TAG or SE synthase.

10. The method of claim 8 wherein the candidate modulator is a positive modulator of a TAG or SE synthase.

11. The method of claim 8 wherein the candidate modulator is a polypeptide.

12. The method of claim 8 wherein the candidate modulator is a defined organic or inorganic compound.

13. The method of claim 1, wherein the yeast cells are of the species Saccharomyces cerevisiae.

14. The method of claim 13, wherein the yeast cells are of a S. cerevisiae strain impaired of neutral lipid synthase production.

15. The method of claim 14, wherein the yeast cells are of a quadruple knock-out S. cerevisiae strain.

16. The method of claim 15, wherein the S. cerevisiae strain is quadruple knock-out dga1, lro1, are1 and are2.

17. The method of claim 1, wherein the neutral lipid synthase is a TAG synthase, a SE synthase or a wax ester synthase.

18. The method of claim 15 wherein the neutral lipid synthase comprises diacylglycerol acyltransferase 1 (DGAT1), diacylglycerol acyltransferase 2 (DGAT2), phospholipid-diacylglycerol acyltransferase (PDAT), acyl-CoA: cholesterol acyltransferase (ACAT), or lecithin:cholesterol acyltransferase (LCAT).

19. The method of claim 14, wherein the method is used for high throughput screening.
